(19) United States
(12) Patent Application Publication
(10) Pub. No.: US 2014/0127776 A1
(43) Pub. Date: May 8, 2014

(54) METHOD USING IMMOBILIZED ALGAE FOR PRODUCTION AND HARVEST OF ALGAL BIOMASS AND PRODUCTS

(71) Applicant: Gaston Picard, Quebec (CA)

(72) Inventor: Gaston Picard, Quebec (CA)

(73) Assignee: AL-G TECHNOLOGIES INC., Levis Quebec, QC (CA)

(21) Appl. No.: 14/125,889

(22) PCT Filed: Jun. 13, 2012
(86) PCT No.: PCT/CA2012/050397

§ 371 Date: Dec. 12, 2013

Related U.S. Application Data
(60) Provisional application No. 61/496,171, filed on Jun. 13, 2011.

(51) Int. Cl. C12N 11/10; C12R 1/89

(52) U.S. Cl. CPC C12N 11/10; C12R 1/89
USPC 435/178

(57) ABSTRACT

Compositions, articles, apparatus, methods and systems are provided for the growth of algae immobilized on a support in a gaseous environment supplying access to sources of carbon dioxide and light, and for subsequent harvesting and biomass processing.

### CROSS-REFERENCE TO RELATED APPLICATIONS

This application claims priority to U.S. Provisional Application No. 61/496,171, filed on Jun. 13, 2011, and entitled “COMPOSITIONS, ARTICLES, APPARATUSES, METHODS AND SYSTEMS RELATING TO ALGAE BIOMASS”, the entire contents of which is incorporated herein by reference for all purposes.

### FIELD

The present invention relates to a compositions, articles, apparatuses, methods and systems relating to algae biomass and uses of algae biomass.

### BACKGROUND

Worldwide increase demand for fossil oil has resulted in two major problems: price increases and increasing air pollution by released carbon dioxide (CO₂), carbon monoxide (CO), and other noxious gases in the atmosphere. Recently, the growth of unicellular algae in wet culture has been proposed to produce algal biomass, containing lipids that can be transformed into commercially useful biodiesel, or biomass convertible to alcohols. It has been reported that there is an average value of 23% for lipid content of 55 investigated microalgae species. The carbon balance is improved when biodiesel from algae is used rather than from fossil oil, since algal biomass consume atmospheric carbon to produce their lipid content, with natural sun light energy.

Commercialization of known processes for making algal fuels suffers from various problems. Open pond production systems may be practical in some geographic areas, but not for others. Known enclosed photobioreactors with high photosynthesis efficiency are being developed and evaluated, but appear far from commercialization. Known growth and harvesting processes appear energy intensive, water intensive, and expensive, leaving a desire for a solution that addresses one or more of these shortcomings that would lead to scalable, cost-effective means for producing algal biomass and resulting fuels.

### SUMMARY

The present disclosure describes several embodiments and aspects or features of the embodiments relating to compositions, articles, apparatuses, methods and systems relating to algae biomass and uses therefor.

In one aspect, the disclosure is a method for growing and harvesting algae biomass. The method includes applying algae cells to a substrate for growth in or on the substrate. The substrate and algae can be in a gaseous environment that includes carbon dioxide and water to support growth of the algae including to hydrate the algae. Liquid can be applied to the substrate to further hydrate the algae. Nutrient can be applied to the substrate to feed the algae. The application of the liquid to the substrate can be reduced for a period of time before applying the nutrient to the substrate, and can be reduced for a period of time after applying the nutrient to the substrate. Light can be applied to the algae and the substrate to support growth of the algae.

Other embodiments and aspects or features thereof including structure, composition, methodology, and means that carry out the above-described method embodiment. Further while multiple embodiments with multiple elements or aspects are disclosed, still other embodiments, elements, and aspects of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic diagram of an embodiment of an article, apparatus, method and system of the disclosure.

FIG. 2 is a schematic illustration of an embodiment of a substrate clamp that provides a liquid reservoir.

FIG. 3 is schematic illustration of an embodiment of a substrate having holes therethrough.

FIG. 4 is a schematic illustration of an embodiment using unwinding and winding rollers.

FIG. 5 is a schematic illustration of an embodiment similar to the embodiment shown in FIG. 4.

FIG. 6 is a schematic illustration of an embodiment using a continuous loop.

FIG. 7 is a schematic illustration of an embodiment similar to the embodiments shown in FIGS. 1 and 6.

FIG. 8 is a schematic illustration of an embodiment similar to the embodiments shown in FIGS. 1, 6 and 7.

### DETAILED DESCRIPTION

Specific embodiments of the present disclosure are described below including composition, article, apparatus, method and system embodiments that are relevant to growing and otherwise processing algal cells and micro algal cells (or algae). These embodiments and their various elements are only examples of the presently disclosed techniques. It should be appreciated that in the development of any such actual implementation, as in any engineering or design project, numerous implementation-specific decisions can be made to achieve the developer's specific goals, such as compliance with system-related and business-related constraints, which can vary from one implementation to another. Moreover, it should be appreciated that such a development effort might be time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there can be additional elements other than the listed elements, and is not intended to mean that each of the included element is essential. Additionally, it should be understood that references to “one embodiment” or “an embodiment” of the present disclosure are not intended to be interpreted as excluding the existence of additional embodiments that also incorporate the listed elements.

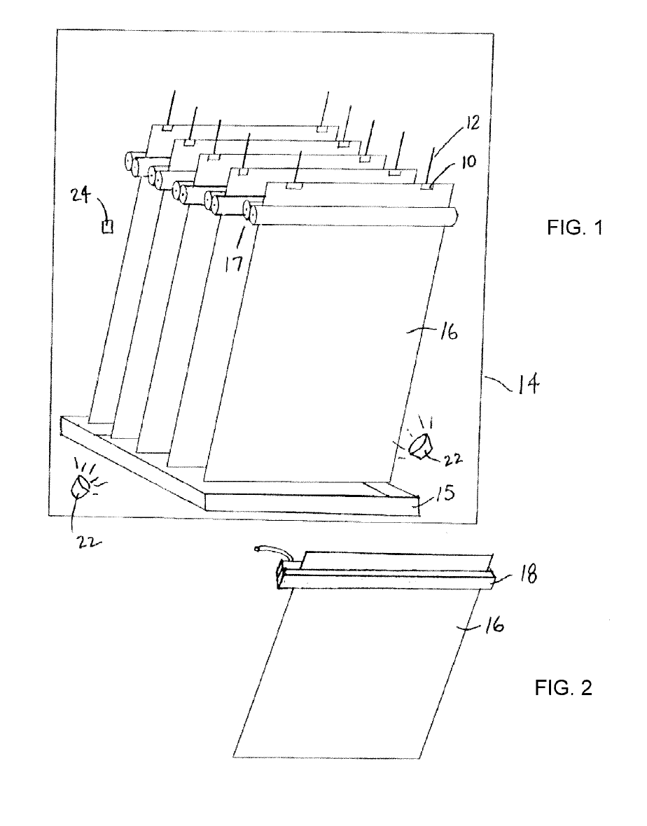

FIG. 1 illustrates an embodiment including suspension members 10, suspension holders 12, an enclosure 14 or greenhouse providing a gaseous environment, a biomass container 15, sheets of algae growth substrate 16 suspended within the enclosure 14 by the suspension members 10 and suspending holders 12, and harvesting devices 17 for transferring algae that has grown on the substrate 16 to the biomass container 15. The suspension members can be wires or cables, and the suspension holders can be clips that hold the sheet is a substantially vertical position while the algae grows on the sheets 16.

The algae can be microalgae and macroalgae. Examples of microalgae include diatoms (Bacillariophyceae), green algae (Chlorophyceae), red algae (Rhodophyceae), yellow-green algae (Xanthophyceae), golden algae (Chrysophyceae), brown algae (Phaeophyceae), and Euglenoids. Two specific microalgaes are Scenedesmus obliquus and Chlorella vulgaris.

As an alternative to the suspension holders 12, a single suspension holder 18 as shown in FIG. 2 can hold or compress a larger surface area on both sides of the sheet 16. In addition, the holder 18 can include an internal reservoir that can receive liquid (e.g., water and/or a nutrient composition) and provide that liquid to the clamped substrate 16 such that the liquid can flow with gravity down the substrate to wet the algae cells, provide them nutrients, or both.

Using one or more of the previously noted approaches can, for example, be used to provides an algae-growing surface area of 80 square meters or more for each square meter of floor space of a greenhouse 14. Groups or modules of, for example, ten substrates 16 can be employed. In a greenhouse that lets in natural sunlight, the substrates can be connected to a device (not shown) that can rotate the sheets to increase the amount of sunlight that contacts the algae on the substrate 16.

Various approaches can be used for applying algae cells to the substrate 16. One approach involves dipping the substrate 16 into a container of algae cells (not shown). Another approach involves spraying the algae/alginate suspension onto the substrate 16 (shown later herein). The algae cells, when applied, can be suspended in an alginate or other gel, or can be wetted with water or another liquid such that the algae adheres to and/or is held on and/or in the substrate 16. Algae can instead be dry or relatively dry when applied to the substrate 16 and subsequently wetted once on and/or in the substrate 16.

Nutrients can be added, for example, 1, 2, 3 or 4 times each day using a spike approach. Nutrients can be applied to the substrate and/or algae within a mist or spray of a water-based nutrient composition. Instead or in addition, a nutrient composition can be applied to the suspended substrate 16 by flowing it from the upper portion of the suspended substrates 10 to the lower portion using gravity. Prior to and after the application of nutrients, the above-disclosed provision of water to the algae can be temporarily discontinued to increase the absorption of the nutrients by the algae.

The amount of the macronutrients and micro nutrients in the feeding nutrients solution can be applied to enable or even optimize cells growth and division. Some of the nutrients include carbon (C), nitrogen (N), phosphorous (P) and potassium (K). Carbon can be provided from carbon dioxide in the air or dissolved in water. Nitrogen can be provided by commercially available ammonium sulfate or ammonium nitrate. Phosphorous can be provided by commercially available phosphates or orthophosphates. Potassium can be provided by commercially available potassium sulfate, potassium chloride or potassium nitrate. These elements can be provided or prepared with specific ratios such as C:N:P of 200:10:1 or 300:5:2 depending on the growth conditions of each species. The ratio of N:P:K can also vary from one group of algae to another group. For example, the ratio can be 4:2:2 or 5:2:1.

In addition, elements such as copper, zinc, molybdenum, cobalt, magnesium, manganese, iron and other elements can be added to suit the selected algae species. The nutrient removal from the added nutrients solution can be followed by a monitoring of the residual nitrogen source leaching from the microalgae growth substrate 16.

As noted herein, the nutrients can be provided intermittently, for example, using a spike approach, rather than continuously to benefit the lipid synthesis process and/or algal cell production. The time between provision of nutrients can be, for example, be one hour, six hours, twelve hours, or a shorter duration.

Though the algae is grown in the gaseous environment provided within the enclosure, the algae can be provided with sufficient water during the algal growth. In addition to the approach disclosed above using a reservoir to irrigation the substrate 16, water can be added by misting or spraying liquid water onto the substrate 16 or to the upper portion of the substrate 16 such that gravity causes the misted or sprayed water to travel to and irrigate the lower portion of the substrate 16. Instead or in addition, the environment around the sheets 10 can have a high relative humidity, such as 80% or higher or lower, to maintain or add a desired amount of water to the algae on the substrate 16. The amount of water provided to the algae can depend on the amount of water lost from the algae or the greenhouse by evaporation. The delivery of water in a liquid phase and the delivery of water in a gaseous phase can be coordinated to provide sufficient water and, if desired, little or no more than a sufficient amount, which results in reduced waste of water and/or in reduced blockage of light energy and/or carbon dioxide intended for delivery to the algae. Water applied but not used by the algae, such as water that drips off the substrates 16 or condenses in the greenhouse, can be recaptured, filtered if desired, and reused.

During the algal growth, the carbon dioxide concentration can be increased to above the normal concentration in air. For example, the carbon dioxide concentration can be five, ten or more times that of the carbon dioxide concentration in air. Further, one concentration can be 6000 parts per million during a portion of the growth period in which light is applied to the algae and a lesser concentration is used during the portion of the growth period in which less or no light is applied to the algae.

The temperature inside the greenhouse can be controlled, that is, kept substantially constant or varied as desired, or can vary with the conditions outside the greenhouse. Using the disclosed embodiments, the temperature of the gas and liquid contacting or surrounding the algae can be more quickly (and with less energy) raised or lowered than approaches that involve growing algae in a pond or other largely liquid growth environments. Also, the choice of algae can depend on the variation of temperature (and other conditions) of the greenhouse, for example, certain algae grow better at higher temperatures than others. The temperature of the gaseous environment and/or the applied liquids can, for example, be between 10° C. and 35° C. Some species of algae grow at temperatures between 16° C. and 27° C. The temperature can more specifically be in the range of 18-20° C. but can vary.

An example of the timing of adding water and nutrients to the algae is to add water within hours 0-9; add nothing within hour 9-10; apply a nutrient composition (which can include water) within hour 10-11; add nothing within hour 11-12; add water within hours 12-21; add nothing within hour 21-22; apply a nutrient composition within hour 22-23; add nothing within hour 23-24; add water within hours 24-33; add nothing within hour 33-34, and so on. This nutrient composition can be delivered to the algal population by, for example, a spike mist, spray or flow of liquid onto the substrate 16. When desired, the algae or a portion of the algae can be harvested from the substrate 16 as is disclosed in detail herein.

One or both of natural sunlight and artificial light can be used during the algal growth. As noted, the substrates 16 can be moved during the day to make better use of the sunlight. Artificial light can be applied continuously using one or more artificial light sources 22, or the algae can be exposed to one or more light periods and dark periods (less or no light). One example of such periods is a 16-hour light period and an 8-hour dark period.

One source of artificial light are fluorescent light lamps, which can provide full, partial, selected, or combination spectrum(s). Incandescent light can be used, as can light-emitting diodes (LEDs) and high pressure sodium lamps. Some sources can, for example, emit specific wavelengths of blue (400 to 500 nm), green (500 to 600 nm), and red (600 to 780 nm). Artificial light sources can be set in a way that it will irradiate the growing algae on the substrate 16. The position of the sources can be selected to direct the light perpendicularly to the surface of the substrate 16 on which the algae is growing, or can be selected to direct the light more toward the side of the substrate 16. The luminance for algal growth can generally be within the range of 20-400 μmol/m²/sec, and more specifically between 80-140 μmol/m²/sec. As noted herein, period of light exposure and period of no (or reduced) light exposure, that is light periods and dark periods, can be employed with (or without) the use of artificial light sources. The duration of a light-dark cycle can vary from algae group to algae group. One such cycle can include a 12 to 14 hour light period. Other cycles can, for example, include a shorter light period of 5 hours or a longer period of 19 hours. The dark period can be adjusted as well to suit the specific algae being grown and/or other aspects of the growth process including the use of energy.

Further, a red light source such as a red LED can be used to reach the first excited state of chlorophylls a and b. A blue light such as a blue LED can also be used as blue light photons provide about 40% more energy than the red light photons. Because light at other wavelengths may help in the regulation of cell growth and metabolism, light sources of other wavelengths can similarly be employed. The light sources can be flashed to simulate the light/dark cycle to prevent or reduce photoinhibition. Because the flux density and time frequency may affect algae growth rate, each can be set to suit the algae being grown. One specific approach can involve a short duration flashed light (<10 μs) with dark intervals of about 10 times longer duration (>100 μs).

Various approaches are disclosed herein to address energy efficiency as well as the inherent limitation of light availability for photosynthesis due to, for example, blockage of light by upper layers of algae in containers used for wet culture. That is, photoinhibition and low light stress of photosynthesis can decrease algal biomass production. Furthermore, photosynthetic pigments (chlorophylls) can exhibit more optimal light absorption, e.g., but not limited to, at around 440 and 680 nm wavelengths. White light with full spectral coverage cannot be fully absorbed but may be suitable for use as a light source, but in some cases part of the light will be reflected or transmitted as wasted energy. Artificial light sources provided around these two wavelengths can be used for light efficiency for algae growth which can be used.

Light sensors 24 can be used to sense the light and, with for example a programmable controller (not shown), can be turned on and off and/or the intensity and wavelength of the artificial light can be changed depending for example on the sensed light intensity, wavelength, duration of exposure of the light upon (or near) the substrate 16, and/or the desired intensity, wavelength and duration. This control approach can be used to provide both the desired light exposure and more efficient use of energy by using natural light during sunny periods and supplemental artificial light at night, cloudy days, or when different intensity, wavelengths, and durations are desired.

Solar collectors can be used in conjunction with the above-disclosed light sources. That is, natural sunlight can be collected within or outside the greenhouse or enclosure and used to directly power the artificial light sources or to charge batteries that can later power the artificial light sources.

A variety of materials and configurations can be used for embodiments of the substrate 16. The substrate 16 can have a structure and composition that is conducive to the inoculating, growing and harvesting of algae and to withstanding the uses of the substrates 16 disclosed herein. The substrate 16 can be a single material such as a single woven or nonwoven fabric layer, a mesh or grid substrate, An example of a single nonwoven substrate is spunbonded polyester, such as is available from DuPont and other companies. Another example is a spunbonded polypropylene, such as is available from Johns Mansville and other companies. Other nonwovens can be meltblown nonwovens and spunlaced nonwovens.

Alternatively, the substrate 16 can be a combination of materials such as multiple woven or nonwoven fabric layers, a nonwoven fabric layer with a film layer, and a woven or nonwoven layer with a grid layer. A combination of nonwoven layers can be a first layer made of a spunbonded polypropylene or polyester fabric with a second layer made of meltblown fabric. The spunbond can provide the strength with the meltblown providing bulk and a finer open matrix in which more algae cells can reside. A grid material could be similarly provide strength to a combination fabric. The grid could be a nylon grid with spacing, for example, of between 1 and 10 mm.

Instead or in addition to the above noted embodiments of various substrates, the substrate 16 could include a main layer on or in which the algae can grow (such as that disclosed above) and a thin top cover layer such as a thin nonwoven layer to provides protection or containment of or support for the growing algae. A thin bottom layer can be added for further protection, containment or support. Top or bottom layers can also provide strength much like the previously disclosed embodiment. One such three-layer embodiment can be two outer polyester spunbonded nonwoven layers with a layer of viscose nonwoven in between. The viscose nonwoven layer can provide hydrophilicity, as can other nonwoven materials including rayon-based nonwovens and other cellulosic-based nonwovens. The polyester outer layers, being thermoplastic, can be thermally bonded, such as point-bonded to hold the three-layer construction together. Other constructions can provide a similar results, such as a construction using a meltblown nonwoven layer treated with a surfactant in place of or in addition to the previously noted viscose nonwoven layer. Still further, rather than multi-layer substrates, other substrates can be single-layer constructions that include multiple fiber types and/or compositions, such as a mixture of spunbonded fibers, meltblown fibers, thermoplastic fibers, cellulosic-based fibers, and other fiber types and compositions.

The substrate 16 can be hydrophilic, as noted above, which can better enable adherence between the substrate 16 and the water, algae, and/or the nutrient composition. The substrate can also have a pH that is conducive to algae growth, such as a range of between 4.5 to 11. The substrate 16 can be selected to avoid or reduce any toxicity to the algae.

The composition and/or the structure of various embodiments of the substrate 16 can contribute to the significant exposure of light to the algae. The substrate 16 can permit transmission of a significant amount and percentage of light onto, into and/or through the substrate 16 to provide significant amount of light to a larger portion of the algae on or in the substrate 16. Contributions to light exposure includes, for example, the transparency or translucency of the material that makes up the substrate 16 (and, if translucent, the color of the substrate 16 can be white or another light color), and the openness of the material that makes up the substrate 16. One example is a substrate including a white spunbonded polyester fabric. Another example is substrate including a white woven polyester fabric. If a polymeric film layer is used with a woven or nonwoven layer, the polymeric film layer can permit light transmission by being transparent or translucent (and if translucent, the substrate can be white or another lighter color).

The composition and/or the structure of various embodiments of the substrate 16 can contribute to the significant exposure of carbon dioxide to the algae. The openness of the substrate 16, such as a woven or nonwoven fabric, enables the passage of gas such as carbon dioxide into and/or through the substrate 16. As disclosed herein, the concentration of carbon dioxide in the environment surrounding the substrate 16 and algae can be increased to above the normal concentration in air. Though not shown, pure carbon dioxide or a gaseous mixture having a high concentration of carbon dioxide can be flowed onto, into, and/or through the substrate 16, such as from one or more nozzles that are connected to a supply of carbon dioxide. In addition to carbon dioxide, gaseous water can be flowed onto, into or through the substrate 16. Further, the gas that is forced through the substrate 16 of a portion of this gas can be collected with a vacuum device as a means for removing oxygen exhaled by the algae and a means for controlling the gaseous composition in the greenhouse.

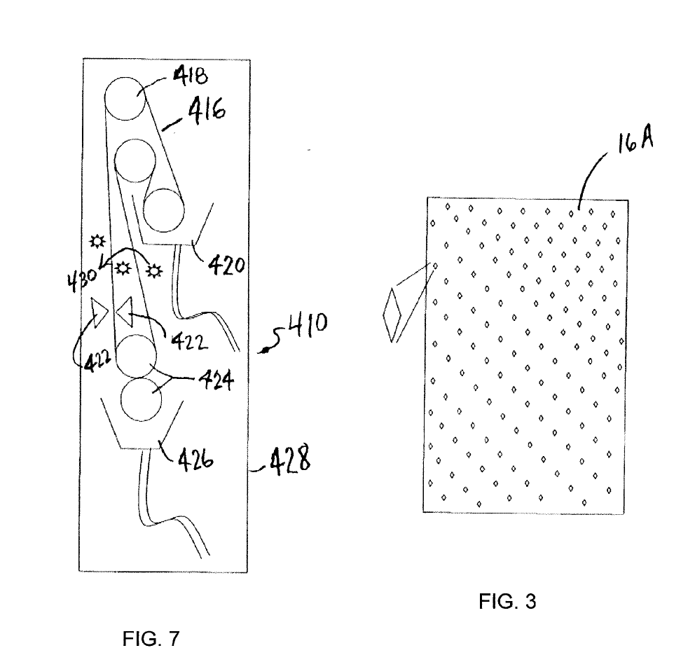

The substrate 16 can have other aspects that are conducive to an aspect of the above-disclosed approaches, such as being conducive to algae inoculation, growth and/or harvesting. One embodiment of the substrate 16A is shown in FIG. 3. Substrate 16A is a nonwoven fabric with holes therethrough. The holes can increase the passage of gas, liquid and sunlight. One shape of the holes can be a diamond shape, although the holes can be circular, oval, square, rectangular or have some other shape. In one embodiment, each hole has dimensions 10.5 millimeters by 3 millimeters (though shown larger in FIG. 3), which equates to a space of 17.4 square millimeters. For example, one embodiment includes 42 holes of this size on a sheet having an area of 58,500 square millimeters, The sizes, shapes, spacing, and other aspects of the holes can be modified as desired.

The liquid to wet the substrate 16 or to be used within the nutrient composition can be water, such as tap water, filtered water, distilled water, deionized water, and/or waste water. Waste water can include certain polluted effluents that allow for or can even contribute to algae growth, which can provide two results: keeping the algae sufficiently hydrated and making use of waste water. Any waste water that drips from the substrate 16 can be in a less polluted condition as a result of absorption of compositions in the water by the algae. To make use of nutrient-rich waste water, the disclosed apparatuses and systems can be located close to a source of such waste water.

The growth period prior to harvesting the cells from the substrate 16 for subsequent processing can be various lengths of time. For example, the first harvest can be three days after inoculation. Successive subsequent harvesting can occur every day, two days or more after the first harvest.

Each harvesting device 17 shown in FIG. 1 is made up of two rollers that press off the substrate 16 a first portion or amount of the algae and leaving a second portion or amount effectively as inoculants for one or more subsequent growths. Though a harvesting device 17 is shown for each sheet of substrate 16, instead, one harvesting device 17 can be used to harvest all or several of the sheets in a greenhouse, and either the harvesting device 17 can be moved from sheet to sheet or the sheets can be transported, such as with a conveyor, to a harvesting device 17. In place or in addition to pressing the algae from the substrate 16, algae can be removed from the substrate by blowing it off using one or more nozzles or air knives (not shown) that direct air or another gas or gas mixture at the substrate 16.

Various embodiments disclosed herein can result in the production of a significant concentration or number of cells reaching 10⁹ cells per square centimeter of substrate, 10¹⁰ cells per square centimeter of substrate, or higher. In addition, the disclosure herein indicates an efficient use of water to produce algae, use of municipal, agricultural, waste effluents for their high nitrogen content. The disclosed systems, apparatuses, methods, articles and composition can also be used in a variety of locations including greenhouses set up on infertile, arid, and/or sloped land.

The approaches disclosed herein can be carried out without having to remove water from the harvested algae, such as using a centrifuge. The harvested concentrated algae can, however if desired, be further concentrated using a centrifuge and/or dried in various ways, for example, by placing the algae into a drying oven or simply leaving the algae exposed to drier gas and/or sunlight to dehydrate the algae. The dehydrated algae can be held in bags, such as polyethylene bags, and stored for later use. Later use can for example include extraction, fractionation, or other isolation of particular parts of the algae such as the lipids, which are disclosed in more detail herein.

One example of the use above-disclosed structures is as follows. A nonwoven substrate was used to grow a mixture of two microalgae, Scenedesmus obliquus and Chlorella vulgaris. The surface area of the substrate was 558 square centimeters, which was inoculated by covering the substrate with a 1% solution of alginate containing the mixture of the two microalgae. The algae cell density was 10⁵ cells per milliliter. The substrate was suspended vertically in a greenhouse that was maintained at a temperature of between 20 and 26 degrees Celsius and at a relative humidity of between 90 and 95%. The light period was 16 hours per day, and the dark period was 8 hours per day. The carbon dioxide concentration was between 500 and 1500 parts per million. Nutrients were delivered to the algae in a water-based nutrient composition. The algae was allowed to grow for 35 days. Growth was monitored daily by weighing the substrate. Harvesting, using the pressing roller approach disclosed above, was carried out every third day. Between 20 and 80 grams of algae were harvested every third day per every square meter of floor space covered by the substrate. No algae cells were added after the initial inoculation showing that the inoculation, growth and harvesting approaches provided a sustainable approach for production of algal biomass.

A similar example involved inoculating and adding water and nutrient to the substrate, and harvesting approximately half of the algae when the algae was estimated to have reached 4×10⁸ cells per square centimeter of substrate leaving the other half as inoculants for subsequent harvests. A subsequent harvest was carried out when the algae was again estimated to have reached 4×10⁸ cells per square centimeter of substrate. This example and the previous example could have been altered to allow more or less growth between harvesting.

There is a variety of other embodiments that can be used in place of or in conjunction with the above-noted embodiments or of elements or aspects of the above-noted embodiments. One such embodiment includes the use of a corrugated or wavy substrate 16A instead of a flat substrate (not shown). The non-flat shape provides a means for increasing the surface area of the substrate. Various shapes of corrugation are possible.

Another embodiment includes applying algae, liquid and/or nutrient to not both sides of the substrate 16, 16A, but to only one of the sides. This can be accomplished with the previously disclosed spray-on approach.

Another embodiment or a further disclosure of prior embodiment includes the lengths of substrate 16 being prepared by applying algae to a longer length of material in ways disclosed herein. The longer lengths can be cut into the shorten lengths shown in the previously mentioned figures.

Another embodiment or a further disclosure of prior embodiment includes applying the algae, liquid and/or nutrient composition to the substrate 16 using an unwinding roller and applying structures disclosed later herein.

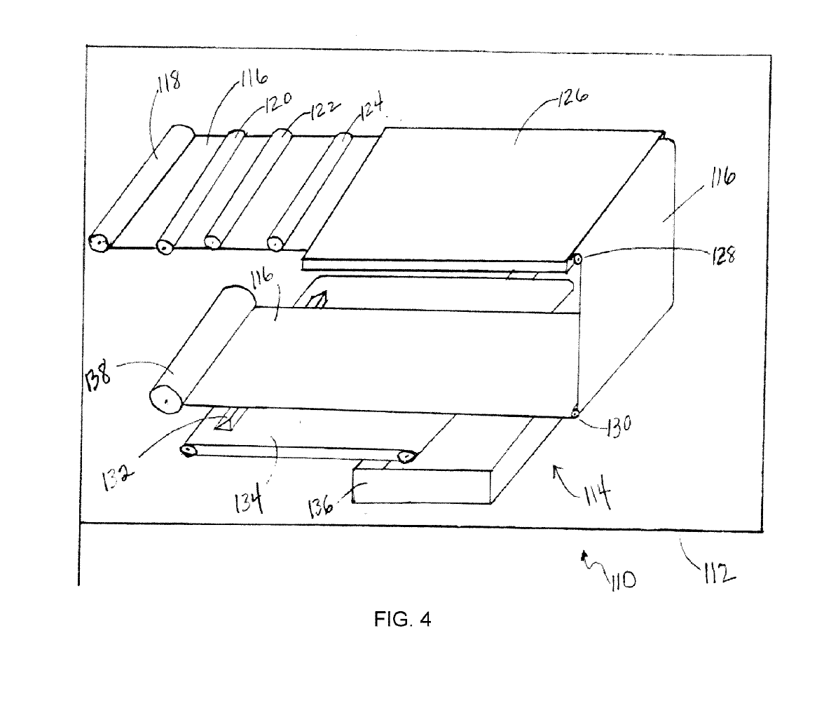

FIG. 4 illustrates an embodiment of an algae growing and harvesting system 110. This embodiment can use one or more of the elements of embodiments disclosed herein. The system 110 can include one or more of the following elements. A system enclosure 112 (like a greenhouse) encloses one or more parts of a growing and harvesting apparatus 114 and a substrate 116 on which algae can be grown. The apparatus 114 in this embodiment includes an unwinding member 118, an algae applicator 120, a liquid applicator 122, a nutrient applicator 124, a treatment device 126, two direction turning members 128, 130, a harvesting device 132 (in this case a stationary mechanical knife), an algae transporter 134, an algae container 136, and a winding member 138. Not shown but referred to and described herein are the algae, liquid, and nutrient.

The system enclosure 112 disclosed above can provide protection for and contain of the one or more parts of the growing and harvesting apparatus 114 as well as other compositions, articles, and apparatuses. The system enclosure 112 can be made of stainless steel or other metals or materials that can stand up to the conditions created by other elements of the system 110. The system enclosure 112 can contribute to maintaining a controlled environment with respect to the algae growing and harvesting. The controlled environment can, for example, include a particular composition and conditions, such as air at room temperature, atmospheric pressure and 80% relative humidity. Instead, the air or another gaseous or gaseous/liquid composition could be at a higher or lower temperature, pressure or relative humidity.

Other gas compositions can be employed, such as a different concentration of carbon dioxide, oxygen, and/or nitrogen, and a higher relative humidity. For example, as previously disclosed, the concentration of carbon dioxide can be many times more than the normal concentration in air, such as five or ten times more or even more. The relative humidity can be increased to above 80% including when algae intakes most of all of the water via the humidity of the environment, that is, lesser or no intake of water from some liquid delivery means.

Further, environment could include primarily gas, for example including gaseous water, and secondarily liquid in form of, for example, a mist or spray of liquid, for example water. Depending on the amount and frequency that a mist, spray or other application of liquid, the relative humidity can be reduced or allowed to drop to, for example, below 80%.

Still further, the composition and/or conditions of the environment can be varied during the growing period such as varying one or more of carbon dioxide, oxygen, nitrogen, and relative humidity. For example if a light period and dark period are employed in a growth cycle, the carbon dioxide (CO₂) density may be adjusted between 300 and 6000 ppm during the light period, and may be adjusted to between 300 and 600 ppm during the dark period.

The various gases can be provided by tanks of each and relative humidity can be provided by a humidifier. Gas density meters and a relative humidity meter can be used to measure and control the environment.

In addition to maintaining the desired gas composition, the controlled environment can keep from or reduce the contact of or interaction with the algae certain materials that can adversely affect the algae, its growth, or other aspects of disclosed systems, compositions, articles, apparatuses, or methods. For example, because certain bacteria can reduce algal growth while certain other bacteria can benefit the algal growth, the inclusion or exclusion of bacteria greenhouse environment can be controlled as desired. Air filtration can be included within a process of controlling the bacteria, as well as other materials.

Further details regarding the enclosure 12 and apparatuses that it can include or interact with to control the environment are disclosed herein, such as air filtration.

The substrate 116 disclosed above can be an article having a structure and composition that is conducive to the growth of algae. For example and as disclosed elsewhere herein, the substrate can be a single material such as a single woven or nonwoven fabric layer, or a combination of materials such as multiple woven or nonwoven fabric layers, a nonwoven fabric layer with a film layer.

The unwind member disclosed above can be an unwind roller 118 that provides means for supplying the substrate 16. The unwind roller 18 can be driven by an electric motor with its rotational speed and/or tension controlled manually or by a programmable controller. The speed of the unwind roller 118 can be coordinated with the speed of the wind-up roller described later herein. The algae applicator disclosed above can be an algae application roller 120 that provides means for applying algae to the substrate 116. As noted the algae cells can be suspended in a gel or another carrier or can be applied within a carrier. Once applied, the algae (described later herein) can remain on the top surface of the substrate 116, move to within the substrate 116, move down near or to the bottom of the substrate 16, or some combination thereof. The algae application roller 120 can be a foam roller that takes or receives algae from a supply of algae and transfer some or all of that algae to the substrate 116. Other means for applying the algae can include one or more sprayers that spray the algae cells (again with or within a carrier) onto the substrate 116. Another means is to extrude or flow an amount of algae cells over the substrate 116. As previously disclosed, algae cells can be applied to one or more sides of the substrate 116.

The liquid applicator disclosed above can be a liquid or wetting roller 122 that provides means for applying a liquid or wetness, for example, water to the substrate 116. The roller can include a hydrophilic foam layer that takes or receives liquid from a liquid supply and transfer liquid to the substrate 116. Instead or in addition to roller 122, other means for application include a misting or spraying device, which are disclosed further herein.

The nutrient applicator disclosed above can be a nutrient application roller 124 that provides means for applying algae growth nutrient to the substrate. The roller 124 can include a hydrophilic foam layer that takes or receives the nutrient composition from a nutrient composition supply and transfers it to the substrate 116. Other means for applying the nutrient include those disclosed above for applying the algae and/or the liquid.

A desired speed at which the substrate 116 is transported can be used to set the distance between various structures of the system 110, including the distance between the algae applicator, liquid applicator, and nutrient applicator so that each is applied when desired. Instead or in addition, the distances between various structures of the system 110 can be adjusted with or without relation to any adjustment to the speed of the substrate 116. Though only one applicator of each type is shown and disclosed above, one or more additional applicators of any type can be provided in the system 110. For example, additional liquid applicators can be used to control the moisture of the algae cells. Also, one or more nutrient applicators can be used if multiple feedings are desired during the algal growth.

The treatment device disclosed above can be a treatment enclosure 126 that provides means for treating the substrate 116 or one or more substances on the substrate to the extent one or more treatments are desired. The treatment enclosure can carry out one or more of a variety of treatments. As one example, the treatment enclosure 126 can prevent or reduce light from getting in such that algae goes through a dark period. Or the treatment enclosure 126 could instead provide a more light than is available or provided outside the enclosure 126. Another example is that the gaseous or gaseous-liquid environment inside the treatment enclosure 126 is different from the environment outside it, such as a different concentration of carbon dioxide or other gas, the humidity, the temperature, other another condition.

The direction turning members disclosed above can be turning rollers 128, 130 each providing means for turning the substrate in a different direction. Other structures for turning the substrate 16 include non-rotating bars or other non-moving members having surfaces over which the substrate 116 can slide.

The harvesting member disclosed above for this embodiment can be a stationary, mechanical knife 132 that provide means for harvesting or removing algae from the substrate 116. The knife can be for example a stainless steel, stationary member with an edge that is positioned relative to the substrate 116 to cut or wipe off algae growing on the substrate. Another structure for harvesting the algae from the substrate 116 is an air knife that applies a sufficient flow or air or other gas onto the algae to remove algae from the substrate 116. As noted and illustrated previously herein, pressing rollers can be used to harvest the algae. Other approaches are brushing the algae from the substrate or vacuuming the algae from the substrate.

The transporter disclosed above can be a conveyor 134 that provides means for receiving the algae and transporting the algae to a location where it can be stored or further processed. The conveyor 134 can include a belt, front and back rollers as well as an electric motor, and a controller for driving one or both of the rollers. Other structures for transporting the algae includes conduit with a sufficient flow rate of air (or other fluid composition) to move the algae as desired.

The container 136 disclosed above provides means for holding the algae pending further processing. This container can be a plastic tube or plastic bag. Rather to be held in the shown container 136, the harvested algae could instead be transported to other container in another location for later processing or immediately to a further processing apparatus or step, such as those disclosed herein. Such further transport means can be provided by a longer conveyor, an additional conveyor, conduit with sufficient air flow to move the algae.

The winding member disclosed above can be a winding roller 138 that provides means for winding up the substrate 116 after part or all of the algae is harvested from the substrate 116. The winding roller 138 is driven by for example an electric motor.

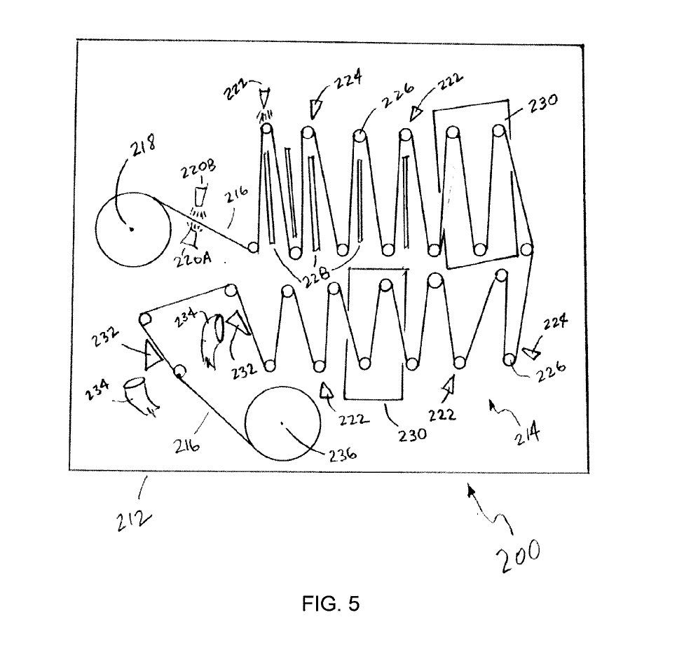

A variation (not shown) of the embodiment shown in FIG. 4 is a system in which inoculation of algae onto the substrate 116 can be done off-line from the growth of the algae. For example, a large roll of substrate 116 can be unwound by the unwind roller, algae cells can be inoculated on to the substrate 116, and the substrate 116 can be rolled up with the winding roller and set aside for a later growth process. The algae can remain sufficient wet to prevent or reduce the loss of cells while the roll of inoculated substrate awaits the growth portion of the process. The subsequent growth portion can involve cutting the substrate 116 into discrete lengths or sheets such as is shown in FIG. 1, or can involve keeping the roll in tack and using the continuous web approach shown in FIG. 4. As shown in FIG. 5, system 200 includes several elements that are similar to the embodiment shown in FIG. 4. This includes a system enclosure 212 that encloses an algae growing and harvesting apparatus 214. A substrate 216 is provided by an unwind roller 218 that holds a jumbo roll of the substrate 216. Upper and lower algae applicators 220A, 220B apply algae cells to the upper portion and lower portion of the substrate 216. Liquid applicators 222 spray or mist liquid, such as water or a composition including water, to the substrate 216. Nutrient applicators 224 spray nutrient composition to the substrate 216. Turning rollers 226 are separated vertically to significantly increase the distance traveled by the substrate 216. Artificial light sources 228 such as fluorescent lamps are shown placed between rollers 226 and the corresponding spans of the substrate 216. Additional and other sources of artificial light can be used. Additional artificial light can be provided turning rollers 226 that are transparent and lighted (not shown). Two sub-enclosures 230 are included to keep light out (or reduced) to provide dark periods for the algal growth (and allow for other condition changes such as gas composition and temperature). Harvesting devices 232 are positioned to remove or harvest algae from the substrate, and the algae is shown gathered through suction members 234 adjacent the harvesting devices 232. The substrate 216, following the harvest, can be wound on to winding roller 236. Because some algae can remain on the substrate 216 following the harvest, the wound roll of substrate 216 on the winding roller 236 can be placed at the unwinding roller 218 and reused or stored for later use. As can be seen, the positioning of the rollers 226 can be used to provide a long length of substrate on which the algae can be grown. One or more of the rollers 226 can be heated or cooled to add or remove heat from the algae on the substrate 216 as desired. Because the rollers 226 contact both surfaces of the substrate 216, the pressure on substrate can be controlled by controlling the tension on the substrate 216, selecting a desired diameter of the rollers 226 and desired substrate pathway, and/or using rollers 226 having a compressible surface material such as foam.

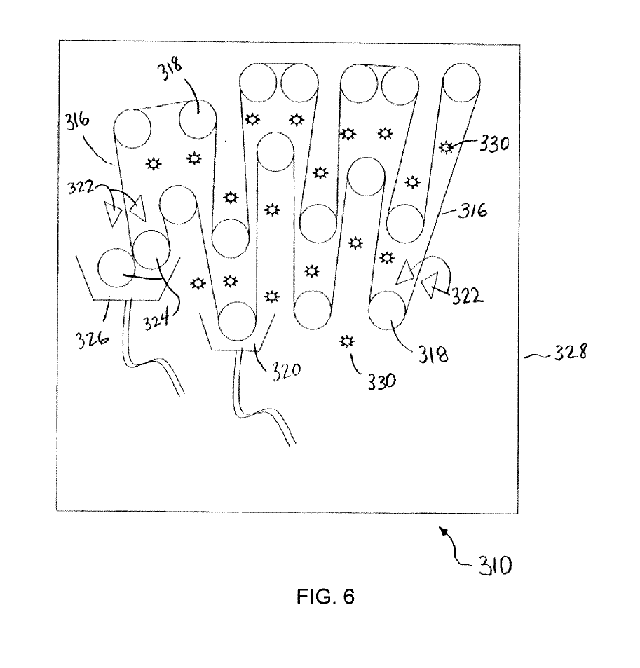

FIG. 6 illustrates an embodiment similar to the embodiments show in FIGS. 4 and 5, except that this embodiment illustrates a system 310 that uses a continuous loop of substrate 316 in place of the substrate 116 and 216 that, as disclosed above, are unwound and wound up. This continuous loop or conveyor system 310 is shown as having a plurality of transport rollers 318, a bath 320, liquid applicators 322, harvesting rollers 324, biomass container, 326, greenhouse enclosure 328, and artificial light sources 330. Various other aspects disclosed above with respect to other embodiments can be included as well, such as the light control, humidity control, gas control, and enclosures that allow for controlling the conditions surrounding the system 310 and/or portions of the system 310.

This system 310 can be operated such that the substrate 316 is started, stopped, slowed, and accelerated as desired to suit the inoculation, growth and harvesting aspects of the system 310. For example, to inoculate the substrate 316, the bath 320 can be filled or partially filled with an algae composition (as disclosed above) and the substrate 316 can be transported through the bath 320, then stopped such that the algae can be exposed to light and gaseous carbon dioxide within the enclosure 328. To wet the algae when desired, the substrate 316 can again be transported such that the liquid applicators can apply (e.g., spray) water or another liquid onto the substrate 316. To feed the algae when desired, the substrate 316 can again be transported such that the liquid applicators 322 can apply (e.g., spray) a nutrient composition onto the substrate 316. In place of or in addition to using the liquid applicators 322, the liquid and nutrient composition can be added to the bath 320 such that transport of the substrate 316 can result in wetting and feeding the algae. After the substrate 316 is transported for wetting and feeding, the substrate can again be stopped for further exposure to light and carbon dioxide in the enclosure 328. The wetting, feeding and growing steps can be repeated as desired. To harvest the algae, the substrate 316 can be transported and the harvesting rollers 324 can be brought together to press off a portion of the algae growing on or in the substrate 316 that can be captured in the biomass container 326 (and removed from there). Following the harvest, the substrate 316 can be reinoculated, rewetted, or refed, some combination thereof, in preparation for subsequent harvests.

A simpler version of the embodiment shown in FIG. 6 is shown in FIG. 7. System 410 of this embodiment is similar to the embodiment shown in FIG. 1, but with a moving, continuous-loop substrate 416 like the substrate 316 provided in system 310. Transport rollers 418, bath 420, liquid applicators 422, harvesting rollers 424, biomass container 426, enclosure 428, and artificial light sources 430 provide similar means as counterpart structures shown in FIG. 6. In this embodiment and other previously noted embodiments, portions of the enclosure 428 that do not permit entry of sunlight, such as the floor, can have a light color such as white to reflect sunlight that has entered the enclosure 428 (and artificial light within the enclosure 428) toward the substrate 416.

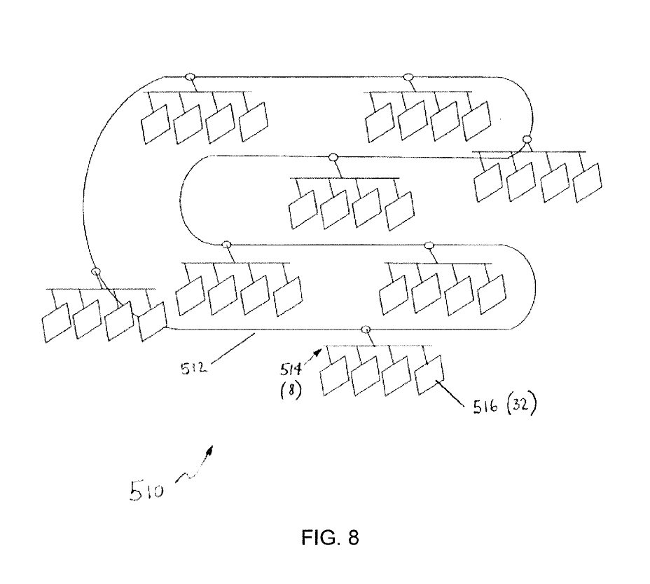

FIG. 8 illustrates another embodiment, which is similar to the embodiments illustrated in FIGS. 1, 6 and 7. This system 510 includes a conveyor 512 with hanging frames 514 each of which is shown suspending four sheets of substrate 516. The system 510 can be used to transport the substrate 516 for one or more reasons including that exposure of light to the sheets of substrate 516 can be controlled, algae can be applied at a station along the path provided by the conveyor (e.g., sprayed or dipped onto the sheets; not shown, but disclosed previously herein), similarly water and nutrient compositions can be applied at other stations (not shown but disclosed previously), and algae can be harvested at one or more other stations (not shown, but disclosed previously). Also, the system 510 can be enclosed within a greenhouse (not shown) that has a controlled environment, including humidity, temperature, gas composition, and the like as previously disclosed.

The above disclosed embodiments and elements thereof cause the algae to be exposed to significantly greater amounts of light and carbon dioxide than if the algae were grown within a body of water or otherwise kept immersed in water. As disclosed, an aspect of this disclosure involves reducing or minimizing the use of water to increase the light and carbon dioxide available to the algae. The disclosed embodiments can be used in conjunction with a carbon dioxide capture means, such as taking carbon dioxide from a combustion, such as a gas furnace used in an adjacent building. Further, the reduced and focused use of water in the disclosure allows for efficient use of nutrients, which reduces cost and can make use of nutrients provided by polluted materials. Still further, some portion of the electricity used in loop approach and the other disclosed approaches (such as the noted steps of transporting; applying algae, water and nutrients; applying light; controlling temperature, gas composition, and other conditions) can be supplied by solar collectors and batteries.

Algal biomass contains 20%-40% protein, 30%-50% lipid, 20% carbohydrate, and 10% other compounds. Depending on the conversion processes, a range of products can be obtained from algal biomass. If a system approach is taken towards the processing of algae biomass, it is possible to maximize the utilization of the biomass for maximum economic and environmental benefits. Biorefining is such a system approach. Biorefining is a concept derived from petroleum refining. A biorefinery uses biomass as feedstock as opposed to fossil resources used in a petroleum biorefinery. The goal of biorefining is to produce a wide range of products such as fuels, materials, chemicals, etc., from one or more biological resources. Because biomass is not a heterogeneous feedstock, several biorefinery platforms such as biological platforms and thermochemical platforms have been proposed. A biorefinery uses a portfolio of conversion and refining technologies and can be integrated with biomass feedstock production. An integrated biorefinery is capable of producing multiple product streams and thus multiple income streams from a single biomass feedstock and, therefore, more economically viable than single product-based production schemes. The heat and energy generated can be used to make the system partially self sufficient in terms of energy.

1. A method for growing and harvesting algae biomass, comprising:
a) applying microalgae cells to at least one suspended substrate sheet;
b) growing said applied microalgae on said substrate sheet in an enclosed humid, gaseous environment that includes carbon dioxide;
c) irrigating the substrate to wet said substrate and microalgae;
d) applying nutrient to the substrate to feed said microalgae;
e) applying light to said microalgae to support growth thereof; and
f) harvesting said microalgae using at least one roller applied to said substrate sheet to press out at least a portion thereof.

2. The method of claim 1, wherein steps c), d) and e) are performed simultaneously.

3. (canceled)

4. The method of claim 1, wherein said steps c), d) and e) are performed in sequential order.

5. The method of claim 1, wherein said steps c), d) and e) are performed out of sequential order.

6. The method of claim 1, wherein in step f), said harvesting is carried out between at least two rollers pressing at least a portion of microalgae from said substrate.

7. The method of claim 1, wherein said microalgae cells are transported on a conveyor during growth.

8. The method of claim 7, wherein said conveyor comprises a plurality of hanging frames for suspending a plurality of substrate sheets, said sheets hanging vertically therefrom.

9. The method of claim 7, wherein said substrate comprises a unitary substrate sheet in the form of a web suspended between transport rollers, said sheet forming a conveyor for transporting said microalgae cells during growth.

10. The method of claim 9, wherein in step f) the harvesting a portion of the microalgae from the substrate is carried out while the substrate and microalgae cells are being transported.

11-12. (canceled)

13. The method of claim 7, wherein said substrate constitutes a continuous loop.

14. (canceled)

15. The method of claim 1, wherein said irrigating is carried out by drip, spray or mist.

16-20. (canceled)

21. The method of claim 1, wherein in step e) the applying of light comprises applying light from non-sunlight light sources.

22. The method of claim 21, wherein in step e) the applying of light comprises applying light to the algae and substrate over at least one light period and further comprises reducing exposure of the algae and substrate to light over at least one dark period.

23-27. (canceled)

28. An apparatus for growing microalgae comprising:
at least one substrate sheet for growing microalgae thereon;
at least one suspension member for suspending said at least one substrate sheet; and
a conveyor for transporting said at least one substrate sheet, said conveyor being adapted for transporting said growing microalgae.

29. The apparatus of claim 28, wherein the conveyor comprises a plurality of transport rollers configured to act as suspension members and adapted for transporting said at least one substrate sheet.

30. The apparatus of claim 28, wherein said at least one suspension member is provided in the form of at least one frame hanging from the conveyor for suspending said at least one substrate sheet.

31. The apparatus of claim 30, comprising a plurality of substrate sheets on said conveyor, said substrate sheets hanging vertically therefrom.

32. The apparatus of claim 28, comprising a unitary substrate sheet forming a web transported between said transport rollers.

33. The apparatus of claim 32, wherein said unitary substrate sheet forms a continuous loop.

34-38. (canceled)

39. A system for growing and harvesting microalgae biomass, comprising:
the apparatus of claim 28 housed in an enclosure, said enclosure providing containment for a humid, gaseous environment that includes carbon dioxide;
an irrigation device for irrigating the substrate and microalgae;
a nutrient applicator for applying nutrient to the substrate to feed the microalgae;
a lighting system to apply light to the substrate to support growth of the microalgae; and
a harvesting device comprising at least one roller to press out at least a portion of said algae from said substrate.

40-41. (canceled)
